(19) United States
(12) Patent Application Publication
(10) Pub. No.: US 2024/0390674 A1
(43) Pub. Date: Nov. 28, 2024

(54) GRIP STRENGTH INCREASING APPARATUS, GRIP STRENGTH INCREASING METHOD, AND PROGRAM

(71) Applicant: NIPPON TELEGRAPH AND TELEPHONE CORPORATION, Tokyo (JP)

(72) Inventor: Arinobu NIIJIMA, Musashino-shi, Tokyo (JP)

(73) Assignee: NIPPON TELEGRAPH AND TELEPHONE CORPORATION, Tokyo (JP)

(21) Appl. No.: 18/691,048

(22) PCT Filed: Oct. 6, 2021
(86) PCT No.: PCT/JP2021/037026

§ 371 Date: Mar. 12, 2024

(51) Int. Cl. A61N 1/36; A61N 1/02

(52) U.S. Cl. CPC A61N 1/36003; A61N 1/025; A61N 1/36031

(57) ABSTRACT

A grip strength enhancement device according to one aspect of the present invention includes a grip motion detection unit that detects a grip motion of a user, and an electrical muscle stimulation (EMS) control unit that controls an EMS presentation unit that presents EMS to a muscle of at least one of an upper arm and a shoulder of the user, and the EMS control unit drives the EMS presentation unit to present the EMS to the muscle of at least one of the upper arm and the shoulder of the user in response to the detection of the grip motion of the user.

### TECHNICAL FIELD

The present invention relates to a technique for assisting a human with grip motions.

### BACKGROUND ART

As a method for enhancing grip strength of a human, a method using an exoskeleton glove is known. For example, NPL 1 discloses an exoskeleton glove for detecting and assisting with a user's grip motion.

### CITATION LIST

### Non Patent Literature

[NPL 1] Pilwon Heo and Jung Kim, “Power-assistive finger exoskeleton with a palmar opening at the fingerpad,” IEEE Transactions on Biomedical Engineering 61.11 (2014): 2688-2697.

### SUMMARY OF INVENTION

### Technical Problem

The exoskeleton glove is a large-scale device and has a high mounting load. The exoskeleton glove also hinders a movable range of a hand and fingers.

An object of the present invention is to provide a grip strength enhancement technique that has a low mounting load and does not hinder a movable range of a hand or fingers.

### Solution to Problem

According to an aspect of the present invention, there is provided a grip strength enhancement device including: a grip motion detection unit configured to detect a grip motion of a user; and an electrical muscle stimulation (EMS) control unit configured to control an EMS presentation unit that presents EMS to a muscle of at least one of an upper arm and a shoulder of the user, in which the EMS control unit drives the EMS presentation unit to present the EMS to the muscle of at least one of the upper arm and the shoulder of the user in response to the detection of the grip motion of the user.

### Advantageous Effects of Invention

According to the present invention, there is provided a grip strength enhancement technique that has a low mounting load and does not hinder a movable range of a hand or fingers.

### BRIEF DESCRIPTION OF DRAWINGS

FIG. 1 is a functional block diagram illustrating a grip strength enhancement device according to an embodiment.

FIG. 2 is a block diagram illustrating a hardware configuration of the grip strength enhancement device according to the embodiment.

FIG. 3 is a diagram illustrating the attachment of the electrodes illustrated in FIG. 2 to a user.

FIG. 4 is a flowchart illustrating a grip strength enhancement method according to the embodiment.

### DESCRIPTION OF EMBODIMENTS

Embodiments of the present invention will be described below with reference to the drawings.

Embodiments relate to a technique for assisting a user with a grip motion. Specifically, the embodiments relate to a technique for enhancing the grip strength of a user by causing a plurality of muscles of the user to contract in cooperation. Embodiments use electrical muscle stimulation (EMS) of presenting electrical stimulation to muscles to cause involuntary contraction in the muscles. Techniques according to embodiments present EMS to the muscle of at least one of the upper arm and the shoulder of the user to cause involuntary contraction in the muscle of at least one of the upper arm and the shoulder of the user in cooperation with voluntary contraction in the user's forearm muscles. The muscle of at least one of the upper arm and the shoulder of the user includes at least one of a biceps brachii muscle of the user, a triceps brachii muscle of the user, and a deltoid muscle of the user. The exemplary embodiment described below presents EMS to the muscles of both the upper arm and the shoulder of the user. The EMS presentation can be achieved by attaching an electrode to a part of a user to whom the EMS is presented. Thus, the mounting load is low and the movable range of the hand and the fingers is not hindered.

### [Configuration]

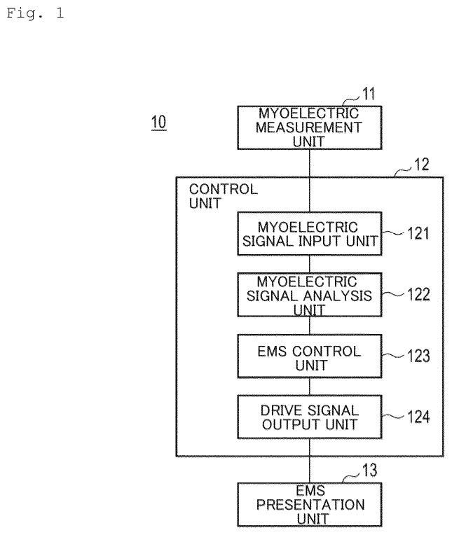

FIG. 1 schematically illustrates a grip strength enhancement device 10 according to an embodiment of the present invention. As illustrated in FIG. 1, the grip strength enhancement device 10 includes a myoelectric measurement unit 11, a control unit 12, and an EMS presentation unit 13.

The myoelectric measurement unit 11 measures myoelectric potential of the user's forearm muscles (for example, a flexor digitorum superficialis muscle) and generates a myoelectric signal indicating the myoelectric potential of the user's forearm muscles.

The control unit 12 controls the EMS presentation unit 13 on the basis of the myoelectric signal obtained by the myoelectric measurement unit 11. The control unit 12 includes a myoelectric signal input unit 121, a myoelectric signal analysis unit 122, an EMS control unit 123, and a drive signal output unit 124.

The myoelectric signal input unit 121 receives the myoelectric signal from the myoelectric measurement unit 11 and sends the received myoelectric signal to the myoelectric signal analysis unit 122.

The myoelectric signal analysis unit 122 corresponds to a grip motion detection unit that detects a grip motion of a user. The myoelectric signal analysis unit 122 analyzes the myoelectric signal in order to detect the grip motion of the user. Specifically, the myoelectric signal analysis unit 122 calculates a root mean square (RMS) of the myoelectric signal over a predetermined time period, and executes detection processing for determining whether or not the grip motion of the user has occurred on the basis of a comparison between the calculated root mean square and a predetermined threshold value at predetermined time intervals. The myoelectric signal analysis unit 122 determines that the grip motion of the user has occurred when the calculated root mean square exceeds a threshold value (for example, 100 μV). The myoelectric signal analysis unit 122 notifies the EMS control unit 123 of the detection of the grip motion of the user when the grip motion of the user is detected.

The EMS control unit 123 controls EMS presented to a user. The EMS control unit 123 sets parameters related to the EMS. For example, the EMS control unit 123 may receive setting information including parameters related to the EMS from a human operator and apply the setting information to the EMS presentation unit 13. Parameters related to EMS include a time length, a frequency, a current, a voltage, and a pulse width. The time length is a parameter indicating the duration for the EMS is presented, and the frequency, the current, the voltage, and the pulse width are parameters for adjusting the intensity of the EMS. For example, the current is set to 10 mA, the frequency is set to 200 Hz, and the pulse width is set to 200 μs.

Further, the EMS control unit 123 drives the EMS presentation unit 13 to present the EMS to the user in response to the detection of the grip motion of the user by the myoelectric signal analysis unit 122. The EMS control unit 123 generates a drive signal for driving the EMS presentation unit 13.

The drive signal output unit 124 outputs the drive signal generated by the EMS control unit 123 to the EMS presentation unit 13. The EMS control unit 123 drives the EMS presentation unit 13 over the set time length. Specifically, the EMS control unit 123 applies the drive signal to the EMS presentation unit 13 via the drive signal output unit 124 over a period from the timing when the grip motion of the user is detected until the time corresponding to the set time length elapses.

The EMS presentation unit 13 presents the EMS to the muscles of the upper arm and the shoulder of the user in order to cause contraction in the muscles of the upper arm and the shoulder of the user under the control by the control unit 12. The EMS presentation unit 13 is driven by a drive signal from the control unit 12. While receiving the drive signal from the control unit 12, the EMS presentation unit 13 generates EMS according to the parameter set by the EMS control unit 123, and presents the EMS to the muscles of the upper arm and shoulder of the user.

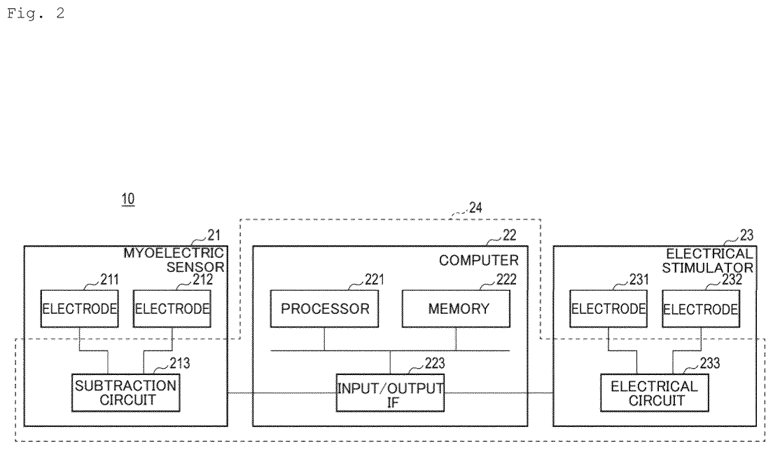

Here, the myoelectric measurement unit 11, the control unit 12, and the EMS presentation unit 13 are collectively referred to as a grip strength enhancement device. As will be described later with reference to FIG. 2, the myoelectric measurement unit 11, the control unit 12, and the EMS presentation unit 13 can be implemented as a plurality of hardware devices separated from each other. The whole may be referred to as a grip strength enhancement system, and the control unit 12 alone may be referred to as a grip strength enhancement device.

FIG. 2 schematically illustrates a hardware configuration example of the grip strength enhancement device 10. As illustrated in FIG. 2, the grip strength enhancement device 10 includes, as hardware components, a myoelectric sensor 21, a computer 22, an electrical stimulator 23, and a mounting member 24. The myoelectric sensor 21 implements the myoelectric measurement unit 11 illustrated in FIG. 1, the computer 22 implements the control unit 12 illustrated in FIG. 1, and the electrical stimulator 23 implements the EMS presentation unit 13 illustrated in FIG. 1.

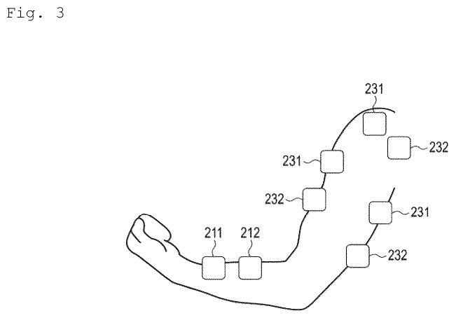

The myoelectric sensor 21 is configured to measure myoelectric potential of a specific muscle of a user to generate a myoelectric signal. As an example, the myoelectric sensor 21 includes a pair of electrodes 211 and 212, and a subtraction circuit 213 connected to the electrodes 211 and 212. The electrodes 211 and 212 may be provided on individual electrode pads. The electrodes 211 and 212 may be provided on a single electrode pad such that they are kept separated from each other by a predetermined distance. As illustrated in FIG. 3, the electrodes 211 and 212 are attached to the forearm of the user to measure the myoelectric potential of the user's forearm muscles. Specifically, the electrodes 211 and 212 are attached to regions of the user's forearm opposite the flexor digitorum superficialis muscle to measure the myoelectric potential of the user's flexor digitorum superficialis muscle. Referring again to FIG. 2, the subtraction circuit 213 outputs a potential difference signal indicating a potential difference between the electrodes 211 and 212 as a myoelectric signal.

The electrical stimulator 23 is configured to apply electrical stimulation to a muscle of a specific part of a user. As an example, the electrical stimulator 23 includes at least a pair of electrodes 231 and 232 and an electrical circuit 233 connected to the electrodes 231 and 232. The electrodes 231 and 232 may be provided on individual electrode pads. The electrodes 231 and 232 may be provided on a single electrode pad such that they are kept separated from each other by a predetermined distance. The electrical circuit 233 generates electrical stimulation (for example, a pulse-like current signal) and applies the electrical stimulation to the user via the electrodes 231 and 232. In the example illustrated in FIG. 3, the electrical stimulator 23 includes three pairs of electrodes 231 and 232, and the three pairs of electrodes 231 and 232 are attached to the upper arm and the shoulder of the user to apply electrical stimulation to the muscles of the upper arm and the shoulder of the user. Specifically, a pair of electrodes 231 and 232 are attached to regions of the user's upper arm opposite the biceps brachii muscle of the user to apply electrical stimulation to the biceps brachii muscle of the user, another pair of electrodes 231 and 232 are attached to regions of the user's upper arm opposite the triceps brachii muscle of the user to apply electrical stimulation to the triceps brachii muscle of the user, and the remaining pair of electrodes 231 and 232 are attached to regions of the user's shoulder opposite the deltoid muscle of the user to apply electrical stimulation to the deltoid muscle of the user.

Referring again to FIG. 2, the computer 22 may be, but is not limited to, a microcontroller. The computer 22 includes a processor 221, a memory 222, and an input/output interface 223. The processor 221 is connected to the memory 222 and the input/output interface 223, and exchanges signals with the memory 222 and the input/output interface 223.

The processor 221 is an example of a processing circuit. The processor 221 includes general-purpose circuits such as a central processing unit (CPU).

The memory 222 stores various types of data and programs executed by the processor 221, such as a grip strength enhancement program. Each program contains a plurality of computer-executable instructions. The processor 221 executes programs stored in the memory 222. The grip strength enhancement program, when executed by the processor 221, causes the processor 221 to perform a series of processes described with respect to the control unit 12. In other words, the processor 221 functions as the myoelectric signal input unit 121, myoelectric signal analysis unit 122, the EMS control unit 123, and the drive signal output unit 124 according to the grip strength enhancement program.

The program may be provided to the computer 22 while being stored in a computer-readable recording medium. In this case, the computer 22 includes a drive for reading data from the recording medium and acquires the program from the recording medium. Examples of the recording medium include a magnetic disk, an optical disc (CD-ROM, CD-R, DVD-ROM, DVD-R, or the like), a magneto-optical disk (MO or the like), and a semiconductor memory. Further, the program may also be distributed through a network. Specifically, the program may be stored in a server on a network, and the computer 22 may download the program from the server.

The input/output interface 223 is an interface for connecting to the myoelectric sensor 21 and the electrical stimulator 23. The processor 221 receives a myoelectric signal from the myoelectric sensor 21 via the input/output interface 223. The processor 221 transmits a drive signal to the electrical stimulator 23 via the input/output interface 223.

The mounting member 24 is a member for mounting the grip strength enhancement device 10 on a user. For example, the mounting member 24 may be a band attached to an upper arm of a user. The subtraction circuit 213 of the myoelectric sensor 21, the computer 22, and the electrical circuit 233 of the electrical stimulator 23 may be provided on the mounting member 24.

The grip strength enhancement device 10 is not limited to a wearable device. Some of the hardware components (for example, the subtraction circuit 213 of the myoelectric sensor 21, the computer 22, and the electrical circuit 233 of the electrical stimulator 23) may be provided on a stationary member.

### [Operations]

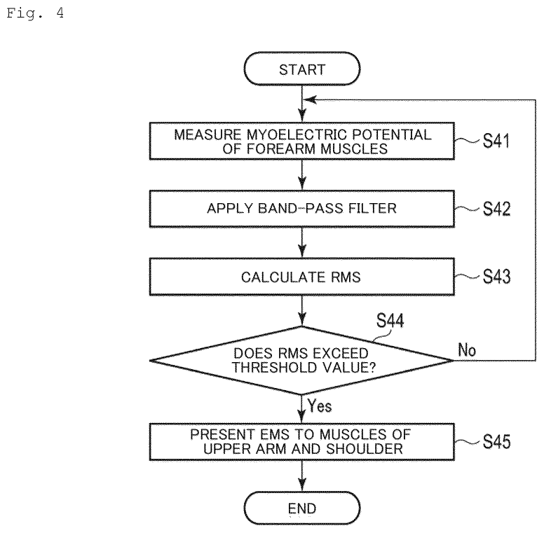

FIG. 4 schematically illustrates a grip strength enhancement method performed by the grip strength enhancement device 10.

In step S41 of FIG. 4, the myoelectric measurement unit 11 measures myoelectric potential of user's forearm muscles. Thus, time-series data related to myoelectric potential of user's forearm muscles is acquired as a myoelectric signal.

In step S42, the myoelectric signal analysis unit 122 applies a band-pass filter having a predetermined pass band (for example, 20 to 450 Hz) to the myoelectric signal output from the myoelectric measurement unit 11 in order to remove noise.

In step S43, the myoelectric signal analysis unit 122 calculates RMS of the myoelectric signal over a predetermined time period. For example, the myoelectric signal analysis unit 122 calculates RMS of the myoelectric signal from a time before a predetermined time to a current time.

In step S44, the myoelectric signal analysis unit 122 determines whether or not the RMS calculated in step S43 exceeds a predetermined threshold value. When the RMS does not exceed the threshold value (step S44; No), the process returns to step S41, and the processes shown in steps S41 to S44 are repeated.

When the RMS exceeds the threshold value (step S44; Yes), the myoelectric signal analysis unit 122 determines that the grip motion of the user has occurred, and the process proceeds to step S45.

In step S45, the grip strength enhancement device 10 presents EMS to the muscles of the upper arm and the shoulder of the user. For example, the EMS control unit 123 generates a drive signal for driving the EMS presentation unit 13, and the drive signal output unit 124 outputs the drive signal to the EMS presentation unit 13. The EMS presentation unit 13 operates in response to the drive signal to present the EMS to the muscles of the upper arm and the shoulder of the user.

### [Effects]

As described above, the myoelectric measurement unit 11 measures the myoelectric potential of the muscle of the upper arm of the user, the myoelectric signal analysis unit 122 performs detection processing for detecting the grip motion of the user on the basis of the myoelectric signal output from the myoelectric measurement unit 11, and the EMS control unit 123 drives the EMS presentation unit 13 to present the EMS to the muscles of the upper arm and the shoulder of the user in response to the detection of the grip motion of the user. This allows for causing involuntary contraction in the muscles of the upper arm and the shoulder of the user in cooperation with the voluntary contraction in the user's forearm muscles. As a result, the user's grip strength can be enhanced. The detection of the grip motion can be achieved by attaching an electrode to the forearm of the user, and the EMS presentation can be achieved by attaching the electrode to the upper arm and the shoulder of the user. Therefore, the grip strength enhancement device 10 is small and light in weight, and has a small mounting load. Further, the movable range of the hand or the fingers of the user is not hindered.

By performing detection processing on the basis of the measurement result of the myoelectric potential of the muscle of the upper arm of the user, it is possible to detect the grip motion of the user with a low processing load. The detection processing may include processing for applying a band-pass filter to the myoelectric signal. Thus, noise included in the myoelectric signal is reduced, and detection accuracy is improved.

Next, an experiment in which it is verified that the grip strength of the user can be enhanced by presenting EMS to the muscles of the upper arm and the shoulder of the user will be described. An experiment was performed on one subject. In the experiment, an electrode pair for myoelectric measurement was attached to a part corresponding to the flexor digitorum superficialis muscle of the subject, three electrode pairs for EMS presentation were attached to parts corresponding to the biceps brachii muscle, the triceps brachii muscle, and the deltoid muscle, and EMS with a current of 10 mA, a frequency of 200 Hz, and a pulse width of 200 μs was presented to each part when the subject exerted grip strength. The experiment was performed according to the following procedure.

1. The subject grips the grip dynamometer for three seconds and measures the maximum grip strength value. The EMS is presented while the subject grips the grip dynamometer.

2. The subject rests for three minutes.

3. The subject grips the grip dynamometer for three seconds without EMS presentation and measures the maximum grip strength value.

4. The subject rests for three minutes.

The above procedure was repeated five times, and the grip strength with EMS presentation and the grip strength without EMS presentation are recorded.

Grip strength with EMS presentation (kgf): 47, 46, 44, 42, 44 Grip strength without EMS presentation (kgf): 37, 44, 40, 43, 45

The average grip strength with EMS presentation was 44.6 kgf, and the average grip strength without EMS presentation was 41.8 kgf.

The above experimental results show that the grip strength can be enhanced by causing contraction in the muscles of the upper arm and the shoulder in cooperation with the muscles of the forearm using EMS. It is also possible to obtain the effect of enhancing grip strength by presenting EMS to at least one of the biceps brachii muscle, the triceps brachii muscle, and the deltoid muscle. By presenting EMS to all of the biceps brachii muscle, the triceps brachii muscle, and the deltoid muscle as in the above experiment, it is possible to further enhance the grip strength.

### Modification Example

The method for detecting the grip motion of the user is not limited to the method using the myoelectric sensor 21. For example, a camera device for obtaining a moving image or a pressure sensor for measuring pressure may be used. In an example of using the camera device, the camera device is provided to photograph a hand of a user or an object gripped by the user, and the grip motion detection unit detects a grip motion of the user on the basis of a moving image output from the camera device. In an example using the pressure sensor, the pressure sensor is built into an object gripped by a user, and the grip motion detection unit detects a grip motion of the user on the basis of a measurement signal output from the pressure sensor. By using a camera device or a pressure sensor, the mounting load is further reduced.

In the above embodiment, the time length for which the EMS is presented is set in advance. Alternatively, the EMS may be presented while the user is performing the grip motion. For example, the myoelectric signal analysis unit 122 calculates RMS at predetermined time intervals even after detecting the grip motion of the user. The myoelectric signal analysis unit 122 determines the timing when the RMS exceeds a first threshold value as the start of grip motion, and determines the timing when the RMS falls below a second threshold value as the end of grip motion, and the EMS control unit 123 drives the EMS presentation unit 13 from the start of grip motion to the end of grip motion. The second threshold value may be the same as or smaller than the first threshold value. By presenting the EMS while the user is performing the grip motion, more tailored assistance to the grip motion of the user is possible.

Note that the present invention is not limited to the embodiments described above and can variously be modified at an execution stage within a scope not departing from the gist of the present invention. In addition, the embodiments may be combined as appropriate, and in such a case, combined effects can be achieved. Furthermore, the above embodiments include various inventions, and various inventions can be extracted by a combination selected from a plurality of disclosed components. For example, even if some components are deleted from all the components described in the embodiments, in a case where the problem can be solved and the effects can be obtained, a configuration from which the components are deleted can be extracted as an invention.

### REFERENCE SIGNS LIST

10 Grip strength enhancement device
11 Myoelectric measurement unit
12 Control unit
13 EMS presentation unit
21 Myoelectric sensor
22 Computer
23 Electrical stimulator
24 Mounting member
121 Myoelectric signal input unit
122 Myoelectric signal analysis unit
123 EMS control unit
124 Drive signal output unit
211, 212 Electrode
213 Subtraction circuit
221 Processor
222 Memory
223 Input/output interface
231, 232 Electrode
233 Electrical circuit

1. A grip strength enhancement device, comprising:
a grip motion detector configured to detect a grip motion of a user; and
electrical muscle stimulation (EMS) control circuitry configured to control EMS presentation circuitry that presents EMS to a muscle of at least one of an upper arm and a shoulder of the user,
wherein the EMS control circuitry drives the EMS presentation circuitry to present the EMS to the muscle of at least one of the upper arm and the shoulder of the user in response to the detection of the grip motion of the user.

2. The grip strength enhancement device according to claim 1, wherein:
the muscle of at least one of the upper arm and the shoulder of the user includes at least one of a biceps brachii muscle of the user, a triceps brachii muscle of the user, and a deltoid muscle of the user.

3. The grip strength enhancement device according to claim 1, wherein:
the grip motion detector detects the grip motion of the user on the basis of a measurement signal output from a myoelectric measurement circuitry that measures myoelectric potential of a muscle of a forearm of the user.

4. The grip strength enhancement device according to claim 3, wherein:
the grip motion detector calculates a root mean square of the measurement signal over a predetermined time period, and determines whether or not the grip motion of the user has occurred on the basis of a comparison between the calculated root mean square and a predetermined threshold value.

5. The grip strength enhancement device according to claim 3, wherein:
the muscle of the forearm of the user includes a flexor digitorum superficialis muscle of the user.

6. The grip strength enhancement device according to claim 1, wherein the grip motion detector detects the grip motion of the user on the basis of a moving image output from a camera device that photographs a hand of the user or an object gripped by the user or a measurement signal output from a pressure sensor built into the object gripped by the user.

7. A grip strength enhancement method comprising:
detecting a grip motion of a user; and
driving electrical muscle stimulation (EMS) presentation circuitry that presents EMS to a muscle of at least one of an upper arm and a shoulder of the user in response to the detection of the grip motion of the user.

8. A non-transitory computer readable medium storing a program for causing a computer perform the method of claim 7.
